(19) United States
(12) Patent Application Publication
(10) Pub. No.: US 2012/0277874 A1
(43) Pub. Date: Nov. 1, 2012

(54) BONE IMPLANTS, SYSTEMS AND METHODS

(76) Inventors: Hansen A. Yuan, Naples, FL (US); Jizong Qi, Beijing (CN); Yong Song, Fremont, CA (US); Jianwen Sun, Beijing (CN)

(21) Appl. No.: 13/509,317

(22) PCT Filed: Nov. 11, 2010
(86) PCT No.: PCT/US2010/056391

§ 371 Date: Jul. 18, 2012

Related U.S. Application Data
(60) Provisional application No. 61/260,336, filed on Nov. 11, 2009.

(30) Foreign Application Priority Data
Jul. 8, 2010 (CN) 201010228241.3

(51) Int. Cl. A61F 2/44; A61B 17/86; B21D 11/14; A61B 17/56; B21C 23/24; A61F 2/28; A61B 17/70

(52) U.S. Cl. 623/17.16; 623/23.61; 606/301; 606/249; 606/86 R; 72/46; 72/371

(57) ABSTRACT

An implantable elastic material configured for use with bone implants is provided with a wire wound in an axially expanded coil form, with the expanded coil formed into a tight mesh. In some embodiments, the wire is formed from a titanium alloy. Methods of manufacturing the implantable material, and implantable devices comprising the material are also disclosed.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to implants, systems and methods for treating various types of orthopedic pathologies, and in particular relates to attachment of implants to bone tissue.

### BACKGROUND OF THE INVENTION

Back pain, particularly in the small of the back, or lumbosacral region (L4-S1) of the spine, is a common ailment. In many cases, the pain severely limits a person's functional ability and quality of life. Back pain interferes with work, routine daily activities, and recreation. It is estimated that Americans spend $50 billion each year on low back pain alone. It is the most common cause of job-related disability and a leading contributor to missed work.

Through disease or injury, the laminae, spinous process, articular processes, facets and/or facet capsule(s) of one or more vertebral bodies along with one or more intervertebral discs can become damaged which can result in a loss of proper alignment or loss of proper articulation of the vertebra. This damage can result in anatomical changes, loss of mobility, and pain or discomfort. For example, the vertebral facet joints can be damaged by traumatic injury or as a result of disease. Diseases damaging the spine and/or facets include osteoarthritis where the cartilage of joint is gradually worn away and the adjacent bone is remodeled, ankylosing spondylolysis (or rheumatoid arthritis) of the spine which can lead to spinal rigidity, and degenerative spondylolisthesis which results in a forward displacement of the lumbar vertebra on the sacrum. Damage to facet joints of the vertebral body often can also results in pressure on nerves, commonly referred to as “pinched” nerves, or nerve compression or impingement. The result is pain, misaligned anatomy, and a corresponding loss of mobility. Pressure on nerves can also occur without facet joint pathology, e.g., a herniated disc.

One conventional treatment of facet joint pathology is spine stabilization, also known as intervertebral stabilization. Intervertebral stabilization desirably controls, prevents or limits relative motion between the vertebrae, through the use of spinal hardware, removal of some or all of the intervertebral disc, fixation of the facet joints, bone graft/osteo-inductive/osteo-conductive material (with or without concurrent insertion of fusion cages) positioned between the vertebral bodies, and/or some combination thereof, resulting in the fixation of (or limiting the motion of) any number of adjacent vertebrae to stabilize and prevent/limit/control relative movement between those treated vertebrae. Stabilization of vertebral bodies can range from the insertion of motion limiting devices (such as intervertebral spacers, artificial ligaments and/or dynamic stabilization devices), through devices promoting arthrodesis (rod and screw systems, cable fixation systems, fusion cages, etc.), up to and including complete removal of some or all of a vertebral body from the spinal column (which may be due to extensive bone damage and/or tumorous growth inside the bone) and insertion of a vertebral body replacement (generally anchored into the adjacent upper and lower vertebral bodies). Various devices are known for fixing the spine and/or sacral bone adjacent the vertebra, as well as attaching devices used for fixation, including: U.S. Pat. Nos. 6,811,567, 6,619,091, 6,290,703, 5,782,833, 5,738,585, 6,547,790, 6,638,321, 6,520,963, 6,074,391, 5,569,247, 5,891,145, 6,090,111, 6,451,021, 5,683,392, 5,863,293, 5,964,760, 6,010,503, 6,019,759, 6,540,749, 6,077,262, 6,248,105, 6,524,315, 5,797,911, 5,879,350, 5,885,285, 5,643,263, 6,565,565, 5,725,527, 6,471,705, 6,554,843, 5,575,792, 5,688,274, 5,690,6306, 022,3504, 805,6025, 474,5554, 611,581, 5,129,900, 5,741,255, 6,132,430; and U.S. Patent Publication No. 2002/0120272.

### SUMMARY OF THE DISCLOSURE

According to aspects of the present invention, an implantable elastic mesh material configured for use with bone implants is disclosed. In some embodiments, the material includes a wire wound in an axially expanded coil form, wherein the expanded coil has been formed into a tight mesh. The wire may be made from a titanium alloy. In some embodiments, at least a portion of the wire has a coating. The coating may include an osteogenic inducer, an osteogenic inhibiter, a medicine, or a combination thereof. In some embodiments, microparticles of a slow release composition are implanted in pores of the material. In some embodiments, the wire has a diameter of between about 0.1 mm and about 0.5 mm. The material may have an axially expanded coil with a pitch that is about three times its nominal diameter.

According to other aspects of the invention, a bone screw pad, a spinous process expander, a vertebral interbody fusion cage, a synthetic nucleus pulposus, or a bone filling block used in osteosynthesis may be provided that includes the material described above.

According to other aspects of the invention, methods of manufacturing an implantable elastic mesh are provided. In some embodiments, the process includes the steps of winding a wire into a coil, winding the coil around a work piece, removing the coil from the work piece, and compressing the coil into an implantable elastic mesh. In some embodiments, the process further includes the step of expanding the coil to a predetermined pitch after it is formed from the wire and before the coil is wound around the work piece. The predetermined pitch may be about three times the nominal diameter of the coil. In some embodiments, the coil is wound around a plate-shaped work piece. In some embodiments, the coil is first wound in one lateral direction along the work piece, then in the opposite lateral direction, and then these steps are repeated until a mesh of required density is achieved. The coil may be first wound in one lateral direction with a first pitch, then in the opposite lateral direction with a second pitch that is about half of the first pitch. A further step may be added in which the coil is removed from the work piece and wound around a mandrel.

In some embodiments of the above described methods, at least a portion of the wire may be coated with an osteogenic inducer, an osteogenic inhibiter, a medicine, or a combination thereof. The coating step may occur before or after the wire is wound into a coil. In some embodiments, microparticles of a slow release composition are implanted into pores of the implantable elastic mesh.

According to other aspects of the invention, the above methods may be used to create all or portions of a bone screw pad, a spinous process expander, a vertebral interbody fusion cage, a synthetic nucleus pulposus, or a bone filling block used in osteosynthesis

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a lateral view of a normal human spinal column;

FIG. 2 is a superior view of a normal human lumbar vertebra;

FIG. 3 is a lateral view of a functional spinal unit;

FIG. 4 is a postero-lateral oblique view of a vertebrae;

FIG. 5 is a perspective view showing a first embodiment of an implantable device constructed according to aspects of the present invention.

FIG. 6 is another perspective view showing the device of FIG. 5.

FIG. 7 is an enlarged cross-sectional view showing a portion of the device of FIG. 5.

FIG. 8 is a perspective view showing another embodiment of an implantable device.

FIGS. 9-13 are various schematic views depicting an exemplary process for creating a mesh washer according to aspects of the invention.

FIG. 14 is a perspective view showing another embodiment of an implantable device.

FIG. 15 is a perspective view showing another embodiment of an implantable device.

FIG. 16 is a partial cross-sectional view showing the device of FIG. 15.

FIG. 17 is a perspective view showing another embodiment of an implantable device.

FIG. 18 is a partial cross-sectional view showing the device of FIG. 17.

FIG. 19 is a fragmentary medial view showing the device of FIG. 9 implanted in adjacent vertebral bodies.

FIG. 20A is a plan view showing an implantable mesh in the form of a circular washer.

FIG. 20B is a side view showing the circular washer of FIG. 20A.

FIG. 21A is a plan view showing an implantable mesh in the form of an elliptical washer.

FIG. 21 B is a side view showing the elliptical washer of FIG. 21A.

FIG. 22A is a plan view showing another implantable mesh body.

FIG. 22B is a side view showing the implantable mesh body of FIG. 22A.

FIG. 23A is a plan view showing another implantable mesh body.

FIG. 23B is a side view showing the implantable mesh body of FIG. 23A.

FIG. 24A is a plan view showing another implantable mesh body in the form of a circular pad.

FIG. 24B is a side view showing the implantable mesh body of FIG. 24A.

FIG. 25A is a plan view showing another implantable mesh body in the form of an elliptical pad.

FIG. 25B is a side view showing the implantable mesh body of FIG. 25A.

FIG. 26A is a plan view showing another implantable mesh body in the form of a square pad.

FIG. 26B is a side view showing the implantable mesh body of FIG. 26A.

FIG. 27 is a lateral view showing an elastic mesh body being used as a synthetic disc between two adjacent vertebrae, and another elastic mesh body being used as an expander between the spinous processes of the vertebrae.

FIG. 28 is a perspective view showing a pair of elastic mesh bodies being used as interbody fusion cages or interbody filling blocks.

### DETAILED DESCRIPTION

Aspects of the invention relate to implantable devices, including implantable prosthesis suitable for implantation within the body to fix, fuse, anchor, restore and/or augment connective tissue such as bone and cartilage, and systems, tools and methods for treating spinal and other pathologies that incorporate use of the implantable devices. In various embodiments, the implantable devices are designed to replace missing, removed or resected body parts or structure. The implantable devices, tools, apparatus or mechanisms may be configured such that the devices or tools can be formed from parts, elements or components which alone, or in combination, comprise the device or tools. The implantable devices can also be configured such that one or more elements or components are formed integrally to achieve a desired physiological, operational or functional result such that the components complete the device. Similarly, tools can be configured such that one or more elements or components are formed integrally to achieve a desired physiological, operational or functional result such that the components complete the tool. Functional results can include the surgical restoration and functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure.

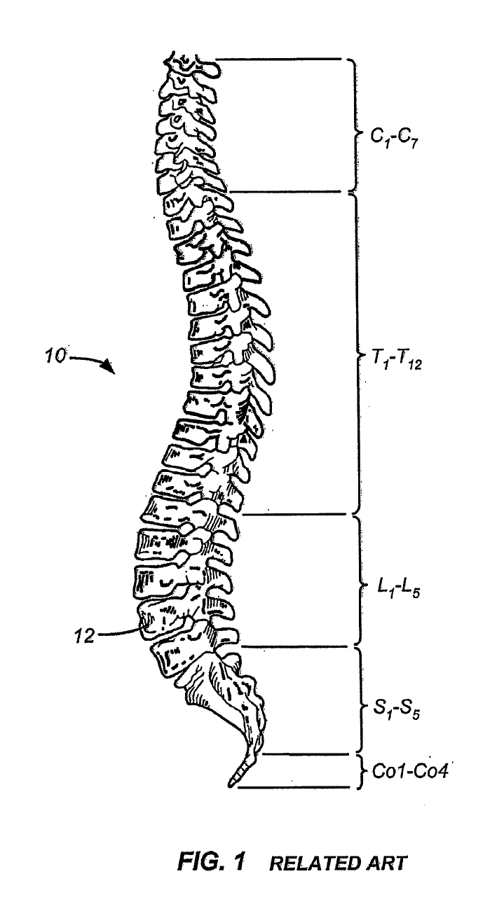

In some embodiments, devices constructed according to aspects of the invention are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

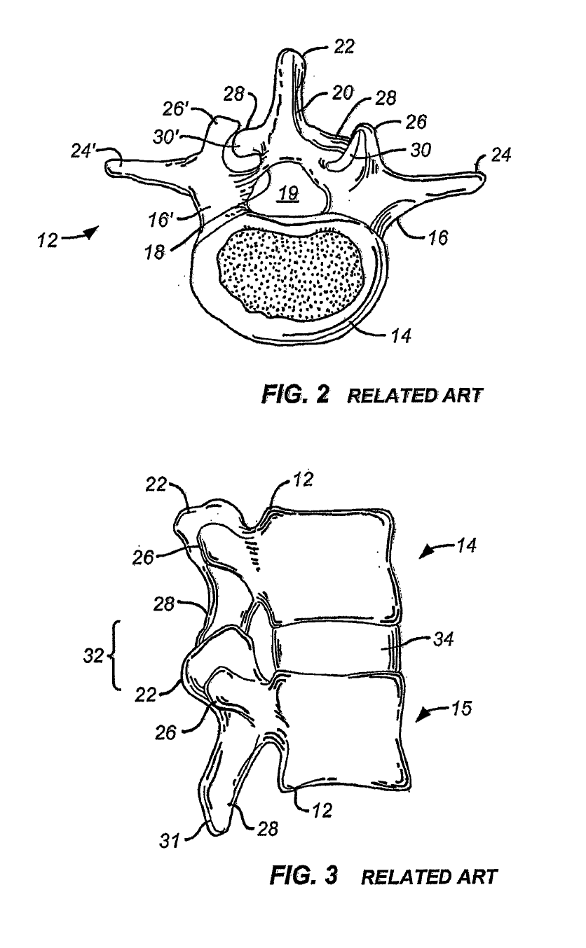

An example of one vertebra is illustrated in FIG. 2 which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles 16, 16′, extend dorsally from each side of the vertebral body 14 to form a vertebral arch 18 which defines the vertebral foramen.

At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles 16 to the laminae 20 is interrupted by the formation of a series of processes.

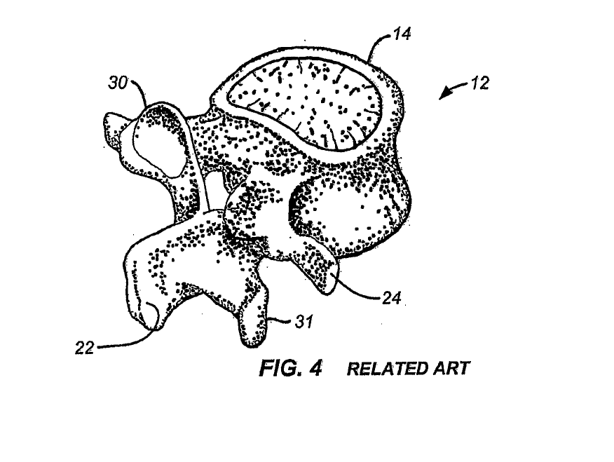

Two transverse processes 24, 24′ thrust out laterally, one on each side, from the junction of the pedicle 16 with the lamina 20. The transverse processes 24, 24′ serve as levers for the attachment of muscles to the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior processes 28, 28′ are oval plates of bone that jut downward on each side. See also FIG. 4.

The superior and inferior articular processes 26 and 28 each have a natural bony structure known as a facet. The superior articular facet 30 faces medially upward, while the inferior articular facet 31 (see FIG. 3) faces laterally downward. When adjacent vertebrae 12 are aligned, the facets 30 and 31, capped with a smooth articular cartilage and encapsulated by ligaments, interlock to form a facet joint 32. The facet joints are apophyseal joints that have a loose capsule and a synovial lining.

As discussed, the facet joint 32 is composed of a superior facet 30 and an inferior facet 31 (shown in FIG. 4). The superior facet is formed by the vertebral level below the joint 32, and the inferior facet is formed in the vertebral level above the joint 32. For example, in the L4-L5 facet joint shown in FIG. 3, the superior facet of the joint 32 is formed by bony structure on the L5 vertebra (i.e., a superior articular surface and supporting bone 26 on the L5 vertebra), and the inferior facet of the joint 32 is formed by bony structure on the L4 vertebra (i.e., an inferior articular surface and supporting bone 28 on the L4 vertebra). The angle formed by a facet joint located between a superior facet and an inferior facet changes with respect to the midline of the spine depending upon the location of the vertebral body along the spine. The facet joints do not, in and of themselves, substantially support axial loads unless the spine is in an extension posture (lordosis). As would be appreciated by those of skill in the art, the orientation of the facet joint for a particular pair of vertebral bodies changes significantly from the thoracic to the lumbar spine to accommodate a joint's ability to resist flexion-extension, lateral bending, and rotation.

An intervertebral disc 34 between each adjacent vertebra 12 (with stacked vertebral bodies shown as 14, 15 in FIG. 3) permits gliding movement between the vertebrae 12. The structure and alignment of the vertebrae 12 thus permit a range of movement of the vertebrae 12 relative to each other. FIG. 4 illustrates a posterolateral oblique view of a vertebra 12, further illustrating the curved surface of the superior articular facet 30 and the protruding structure of the inferior facet 31 adapted to mate with the opposing superior articular facet. As discussed above, the position of the inferior facet 31 and superior facet 30 varies on a particular vertebral body to achieve the desired biomechanical behavior of a region of the spine.

Thus, the overall spine comprises a series of functional spinal units that are a motion segment consisting of two adjacent vertebral bodies, the intervertebral disc, associated ligaments, and facet joints. See, Posner, I, et al. A biomechanical analysis of the clinical stability of the lumbar and lumbrosacral spine. Spine 7:374-389 (1982).

As previously described, a natural facet joint, such as facet joint 32 (FIG. 3), has a superior facet 30 and an inferior facet 31. In anatomical terms, the superior facet of the joint is formed by the vertebral level below the joint, which can thus be called the “caudad” portion of the facet joint because it is anatomically closer to the tail bone or feet of the person. The inferior facet of the facet joint is formed by the vertebral level above the joint, which can be called the “cephalad” portion of the facet joint because it is anatomically closer to the head of the person. Thus, a device that, in use, replaces the caudad portion of a natural facet joint (i.e., the superior facet 30) can be referred to as a “caudad” device. Likewise, a device that, in use, replaces the cephalad portion of a natural facet joint (i.e., the inferior facet 31) can be referred to a “cephalad” device.

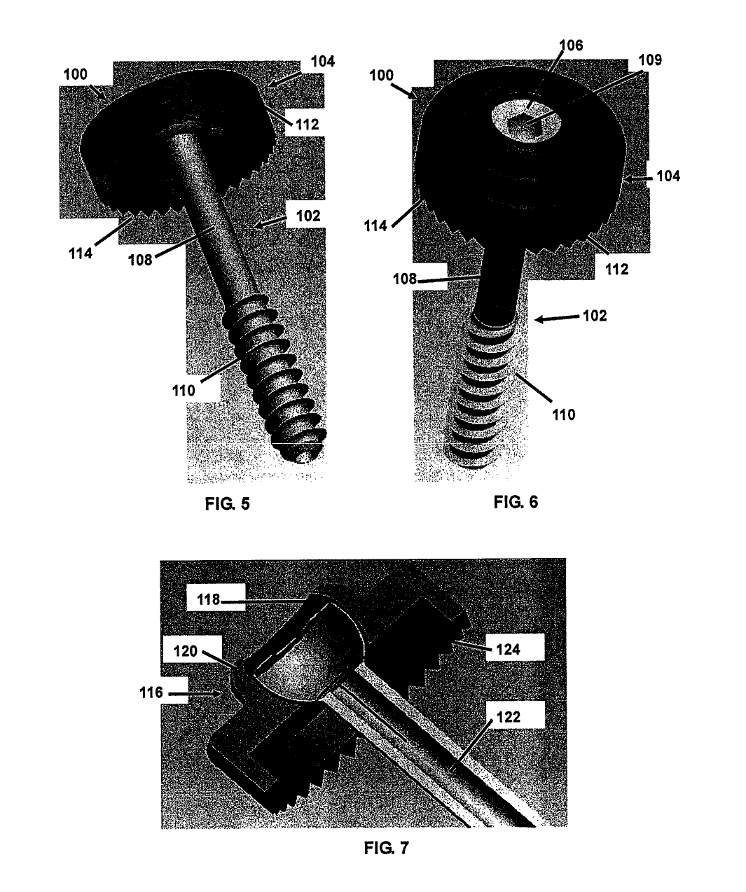

Referring to FIGS. 5-7, an exemplary embodiment of an implantable device 100 constructed according to aspects of the invention is shown. Device 100 includes a bone screw 102 and a cap 104 attached or attachable thereto. Bone screw 102 has a head 106 formed or attached to a shank 108. A keyed socket 109, such as for receiving a hex driver, may be provided in the proximal end of head 106 as shown in FIG. 6. In this embodiment, screw shank 108 includes threads 110 formed on its distal end. In other embodiments, threads may be formed along the entire shank up to the head. In some embodiments, the threads are designed to be self-drilling and/or self-tapping.

In the exemplary embodiment shown, cap 104 is generally disk shaped and includes a distally-projecting flange 112 extending from its outer circumference. One or more teeth 114 may be formed along the distal edge of flange 112 as shown. Teeth 114 may be configured to aid in gripping tissue such as bone, as will be later described. In this embodiment, the proximal face of cap 104 includes a central projection 116. In other embodiments, the entire cap may be dome-shaped.

As best seen in FIG. 7, screw 102 may be pivotably attached to cap 104. In this exemplary embodiment, screw head 106 has a spherical shape and is slidably received within a spherical recess 118 formed in cap 104. Spherical head 106 and spherical recess 118 cooperate to form a ball and socket joint, allowing cap 104 to pivot in any direction relative to screw 102. Overhang 120 may be provided in cap projection 116, such as by swaging after assembly, to pivotably retain cap 104 on screw head 106. In some embodiments, overhang 120 is omitted or is shallow enough to allow assembly and/or disassembly of cap 104 and screw 102 with little or no force. Such an arrangement may be desirable when various sizes of caps 104 may be coupled with various lengths and/or diameters of screws to fit the particular anatomy of each patient, using a surgical kit having a reduced inventory of implantable parts. In other embodiments of the invention, cap 104 and screw 102 may be configured such that they do not pivot relative to one another. In some of these embodiments, cap 104 and screw 102 may be separable, permanently coupled, or integrally formed.

As shown in FIG. 7, screw 102 may be provided with a central lumen 122 extending from socket 109, through shank 108, and out the distal end of screw 102. Lumen 122 may be used to receive a guidewire therethrough, as will be later described. In other embodiments, screw 102 may be solid.

In the exemplary embodiment shown in FIGS. 5-7, cap 104 has an outer diameter of about 15 mm, an overall height of about 5 to 8 mm, and may comprise titanium, a titanium alloy such as Nitinol, or stainless steel. Exemplary screw 102 may be provided in lengths ranging from about 25 to 50 mm, a range of outer shank diameters such as 3.5 mm, 4.0 mm and 4.5 mm, may have an inner lumen diameter of about 1.5 to 1.8 mm, and may be made of titanium, a titanium alloy such as Nitinol, or stainless steel.

In other embodiments (not shown), the distally facing inner surface or the entire cap may have an arced or domed shape. As depicted by arc 124 in FIG. 7, the inner surface may have a radius R as shown. This curvature allows the cap to better conform to certain anatomies, thereby providing more surface contact with the bone. In this exemplary embodiment, arc 124 conforms to the slight convex shape of a facet joint bony surface, as described in more detail below. In some embodiments, the radius R is about 15 mm to 20 mm.

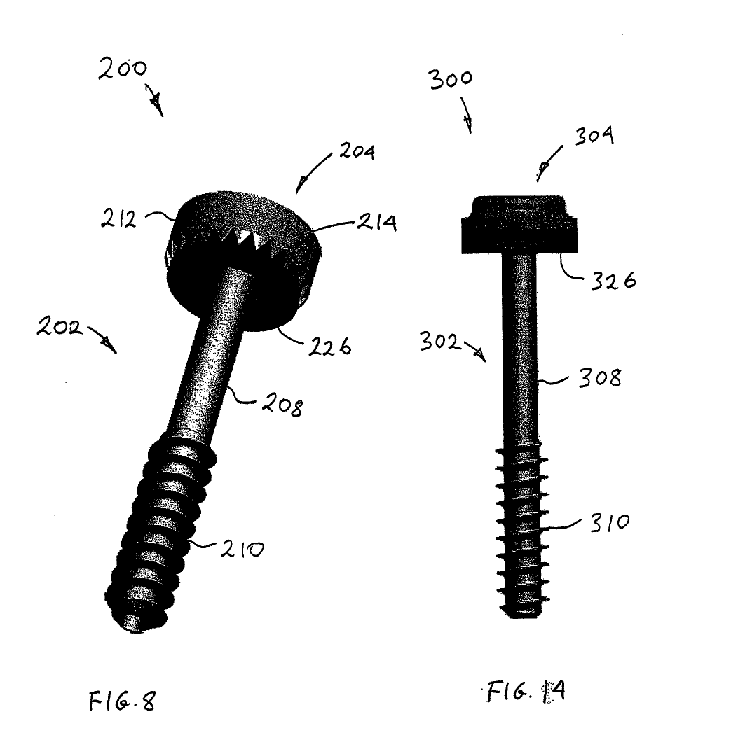

Referring to FIG. 8, another exemplary implantable device 200 is shown. Device 200 is constructed and functions in a similar manner to that of device 100. Device 200 includes screw 202 and cap 204. Screw 202 includes a shank 208 and threads 210. Cap 204 includes a distally-projecting flange 212 with teeth 214 formed on its distal edge.

Device 200 further includes a washer 226. In some embodiments, washer 226 has an outer diameter just small enough to allow it to fit within distally-projecting flange 212 as shown. In other embodiments, the outer diameter of washer 226 may be larger than flange 212, or may be substantially smaller. In some embodiments, washer 226 has an inner diameter substantially larger than the outer diameter of screw shank 208 as shown. In other embodiments, the inner diameter of washer 226 may be nominally the same as the diameter of shank 208. In various embodiments, the thickness of washer 226 is designed to allow washer 226 to be fully recessed within cap 204, generally even with teeth 214, or protruding distally beyond teeth 214 as shown.

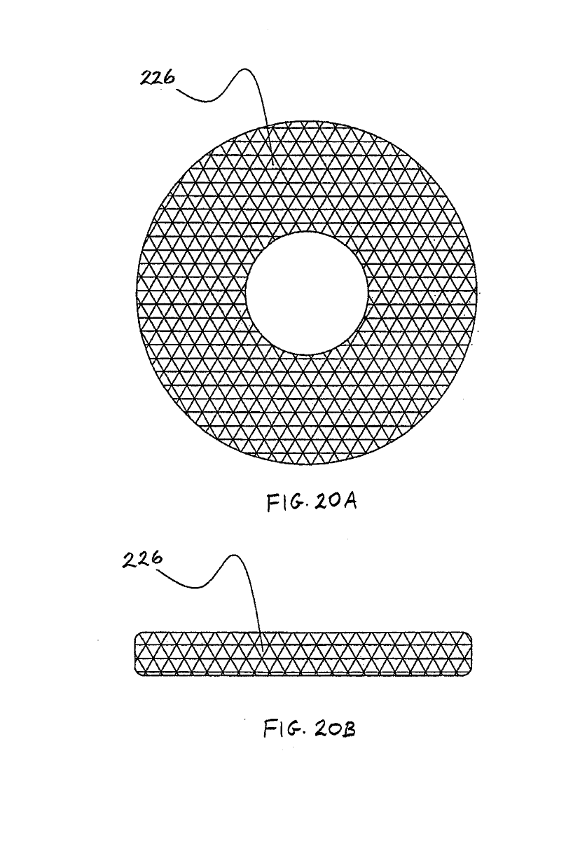

Washer 226 may be formed of a wire mesh, as illustrated in FIGS. 20A and 20B. In some embodiments, the wire mesh comprises titanium, a titanium alloy such as Nitinol, or stainless steel. The wire diameter may be about 0.1 to 0.4 mm depending upon clinical applications.

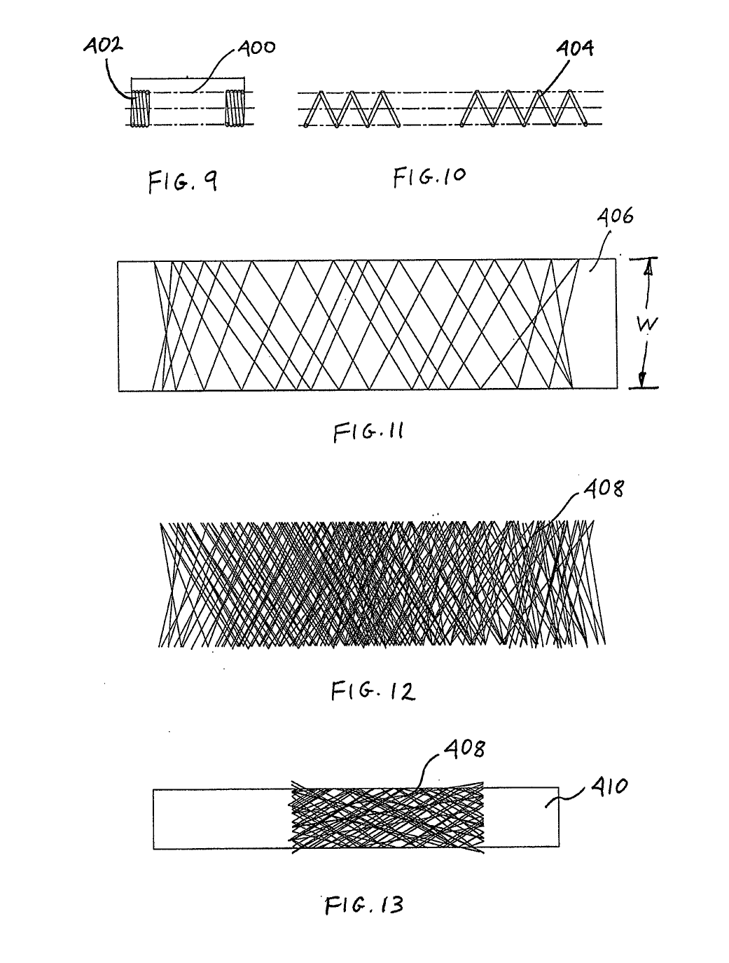

Referring to FIGS. 9-13, an exemplary process for creating a wire mesh according to aspects of the present invention is shown. Referring first to FIGS. 9, 0.1 to 0.4 mm diameter wire is wound around a rod 400 to create an extension spring 402 having its coils close together or touching. In some embodiments, extension spring 402 has a length of about 1 meter or more. Extension spring 402 may then be removed from rod 400 and may be stretched by hand or machine to form a compression spring 404 having its coils separated, as shown in FIG. 10. In some embodiments, adjacent coils of compression spring 404 are stretched to a spacing of 2 to 3 times the diameter of spring 404. In some embodiments, extension spring 402 may be formed on rod 400 with the desired pitch, such that subsequent stretching is not needed. Stretched compression spring 404 may then be wound around a work piece, such as a flat plate 406, as shown in FIG. 11. Plate 406 may have a width W of 30 mm. In the first winding pass, adjacent windings may be spaced apart by 30 mm. In subsequent layers, the distance between windings may be decreased by half that of the previous layer. For example, the first layer may have a distance of 30 mm between windings, the second layer may have a distance of 15 mm, the third layer may have a distance of 7.5 mm, and so on until a unitary, desired density and/or pore size is achieved. As shown in FIG. 12, the wound wire 408 may then be removed from plate 406. As shown in FIG. 13, the flat, wound wire 408 may then be molded around mandrel 410 and formed into a washer shape. In some embodiments, the wound wire 408 may be compressed against mandrel 410. In some embodiments, wound wire 408 may be compressed in a mold to form a desired shape, density, elasticity and/or pore size. In other embodiments, a weaving process may be used to create a mesh from compression spring 404.

In some embodiments, washer 226 is configured to compress as screw 202 is installed into bone. This arrangement allows washer 226 to fill uneven contours in the bone anatomy. In some embodiments, portions of washer 226 may wedge into gaps within or between bones, thereby aiding to secure device 200 in place, and/or provide other advantages such as inhibiting or preventing adjacent bone movement.

In some embodiments, washer 226 serves as a scaffolding to promote tissue growth, such as bony ingrowth from bone contacted by implanted device 200. Such tissue growth can be promoted by coating exterior and/or interior fibers of washer 226 with hydroxyapatite, titanium, and/or calcium phosphate as mentioned above. In some embodiments, washer 226 may include material(s) and/or coating(s) that inhibit tissue ingrowth. Washer 226 may include medicine or other materials and/or coatings that provide therapeutic, diagnosing or imaging benefit(s).

Referring to FIG. 14, another exemplary implantable device 300 is shown. Device 300 is constructed and functions in a similar manner to that of devices 100 and 200. Device 300 includes screw 302 and cap 304. Screw 302 includes a shank 308 and threads 310. In this particular embodiment, cap 304 does not include a distally-projecting flange. Teeth (not shown) may be formed on the bottom surface of cap 304, or the bottom surface may be flat, contoured and/or textured. Device 300 comprises a washer 326 which may be constructed and operated in a manner similar to that of washer 226 of device 200 as previously described. For example, washer 326 may provide a scaffolding to promote tissue growth, as previously described. Because cap 304 of this exemplary embodiment does not have any teeth that protrude distally beyond washer 326, washer 326 may be fully compressed between cap 304 and the bone that screw 302 is inserted into to assist in retaining device 300 in the bone.

Referring to FIGS. 15 and 16, another exemplary implantable device 500 is shown. Device 500 is constructed and functions in a similar manner to that of devices 100, 200 and 300. Device 500 includes screw 502 and cap 504. Screw 502 includes a shank 508 and threads 510. In this particular embodiment, cap 504 has a domed or arcuate shape. As shown in FIG. 16, cap 504 includes an outer set of teeth 512, and an inner set of teeth 514 that are recessed within domed cap 504. The outer teeth 512 and/or the inner teeth 514 may be asymmetrical as shown.

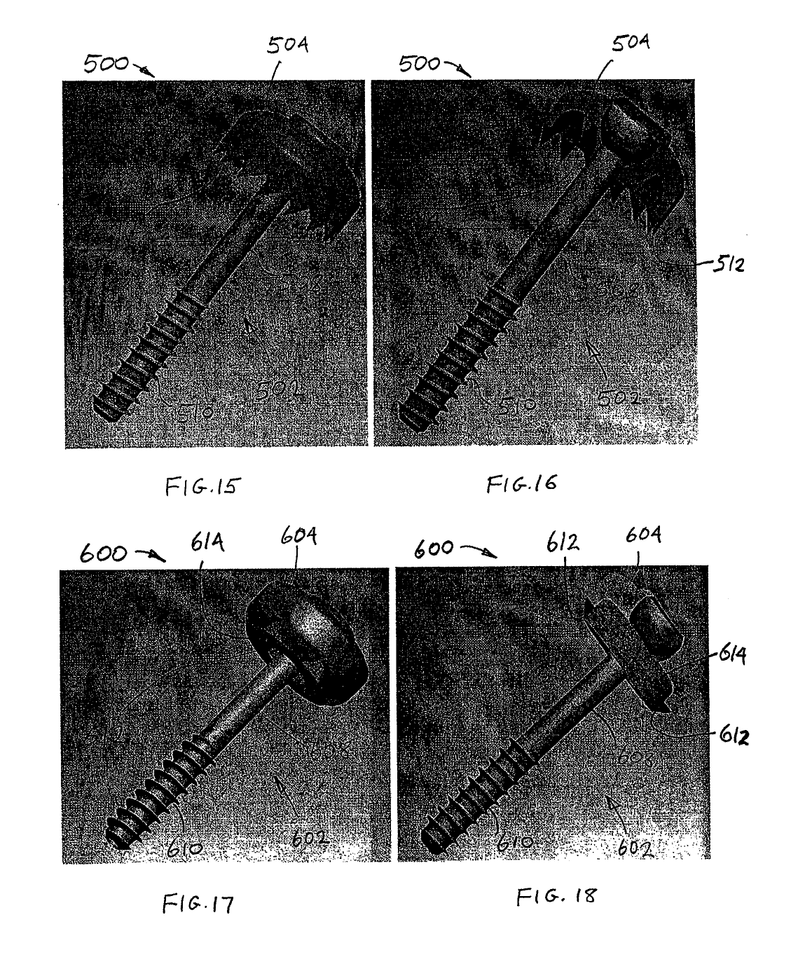

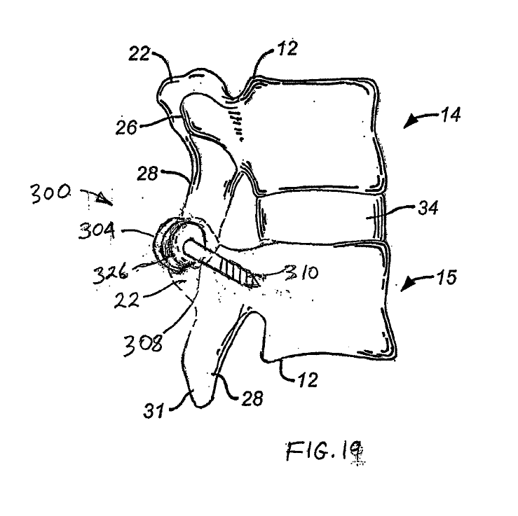

Referring to FIGS. 17 and 18, another exemplary implantable device 600 is shown. Device 600 is constructed and functions in a similar manner to that of devices 100, 200, 300 and 500. Device 600 includes screw 602 and cap 604. Screw 602 includes a shank 608 and threads 610. In this particular embodiment, cap 604 has a set of elongated teeth 612. In other words, each tooth 612 does not come to a point at its distal tip but forms an arcuate distal end that may be sharp in the radial direction but not in the tangential direction. Device 600 also includes a mesh washer 614, similar to previously described mesh washers.

Referring to FIG. 19, an exemplary use of device 300 is shown. In this application, device 300 is used as a facet screw to assist in limiting or preventing relative motion between adjacent vertebral bodies 14 and 15. Screw shank 308 passes through the right inferior facet 31′ of upper vertebral body 14 and through the right superior facet 30′ of lower vertebral body 15. Screw shank 308 is angled in an anterolateral caudal direction toward and/or into the right pedicle 16′ of the lower vertebral body 15. In some embodiments, screw threads 310 engage in pedicle 16′ and draw cap 304 toward the right inferior facet 31′ as shown. As screw 310 is tightened into the bone, mesh washer 326 is compressed by cap 304 against facet 31′, contouring to its non-articulating surface. In this manner, motion between articulating facets 30′ and 31′ is reduced or eliminated. An additional mesh washer or mesh material (not shown) may be placed between articulating facets 30′ and 31′ to further stabilize and/or fuse the two bone portions together.

In some embodiments of the inventive implanting method, a device such as 100, 200, 300, 500 and/or 600 is placed through the facet joints 32 on each side of adjacent vertebral bodies 14 and 15 at one or more levels of the spine. In other embodiments, a device 100, 200 or 300 is placed on only one side. For example, a rod stabilization system may be placed on one side of the vertebral bodies and a fusion cage placed between the vertebral bodies. Instead of another rod system, a device such as 100, 200, 300, 500 or 600 is then placed on the opposite side to prevent excessive trauma while further stabilizing the vertebral bodies.

In some embodiments, a device without teeth, such as device 300, is used to secure the lower spine, such as at level L5-S1 and L4-L5, while a device having teeth, such as device 100 or 200, is used at higher levels of the spine.

Devices 100, 200 and 300 may be implanted with a minimally invasive procedure. In some embodiments, an incision may be made adjacent the spine and a guidewire may be inserted along the desired trajectory through the facet joint. Imaging, such as fluoroscopy or x-ray, may then be used to confirm proper placement of the guidewire. A canulated device 100, 200, 300, 500 or 600 as previously described, may then be placed over the guidewire and screwed into place through the facet joint. In some embodiments, a cannulated drill bit and/or other bone cutting device(s) may be placed over the guide wire prior to the placement of the implanted device to form a hole through the bone for receiving the device.

Additional details of methods, tools, systems and devices for immobilizing a facet joint as described above may be found in U.S. patent application publication no. 2008/0147079 entitled Guidance System, Tools and Devices for Spinal Fixation.

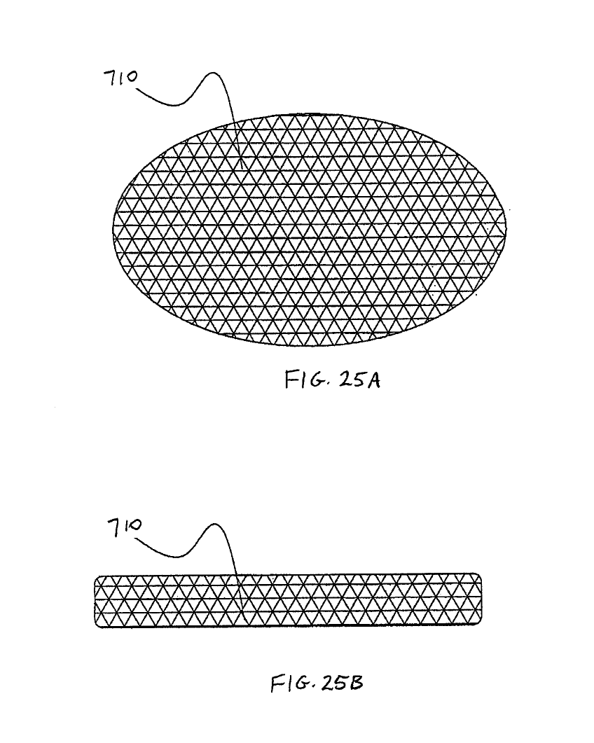

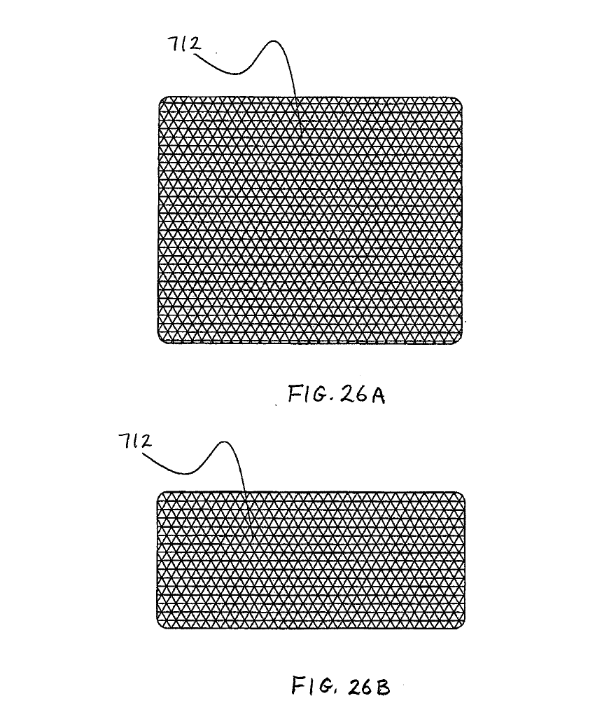

In addition to stabilizing a facet joint, the devices and materials described herein may also be used in other orthopedic applications. For example, devices having at least one wire mesh washer or spacer may be used to conform to flat or contoured bone structures other than facet joints, such as with interspinous spacers and/or with intervetebral cages. Examples of these devices are shown in FIGS. 20A-28, and are subsequently described in more detail. In some embodiments, the wire mesh provides scaffolding for tissue ingrowth. The wire mesh can also be used in sheet form (i.e. not in the shape of a washer) between other implantable devices and bone. The wire mesh may again serve to fill gaps in the bone, help secure the device, prevent or inhibit motion of adjacent bone, and/or provide a scaffold for tissue ingrowth. The elastic mesh may also be used at one or both endplate surfaces of a total cervical or lumbar disc device, and also with some artificial nucleus devices for biological fixation.

Since the previously described implantable elastic material is formed from wire wound into spiral spring, the elasticity and hardness of the material and of devices made from it can be controlled in the molding process based on changes in the pitch of the spiral spring, the density of the mesh and compression used in the molding process in order to meet practical requirements. Additionally, the material has excellent plasticity and can fully conform to other surfaces due to the properties of the wire itself. The pores of the material provided by aspects of this invention provide more space and support for osteoanagenesis and can facilitate rapid bone fusion.

When the elastic mesh disclosed herein is used in orthopedic surgery, an osteogenic inducer coating, an osteogenic inhibitor coating, or a medicine coating may be applied to the wire to facilitate bone growth and fusion or to prevent the over-growth of the bone. The coating may be applied by spraying or another coating process. For instance, an even layer of active factor(s) such as bone growth factors or inhibitors (proteins, peptides, hormones etc.) or medicines (antibiotics, etc.) may be applied on the surface of the elastic mesh, or slow release microparticles of the above substances may be implanted in the pores of the mesh. Bone fusion inducers such as calcium phosphate or hydroxyapatite may be coated on the surface of the elastic mesh material. The loading or coating may be done before or after the winding and molding process of creating the elastic mesh material, or as an intermediate step during the process.

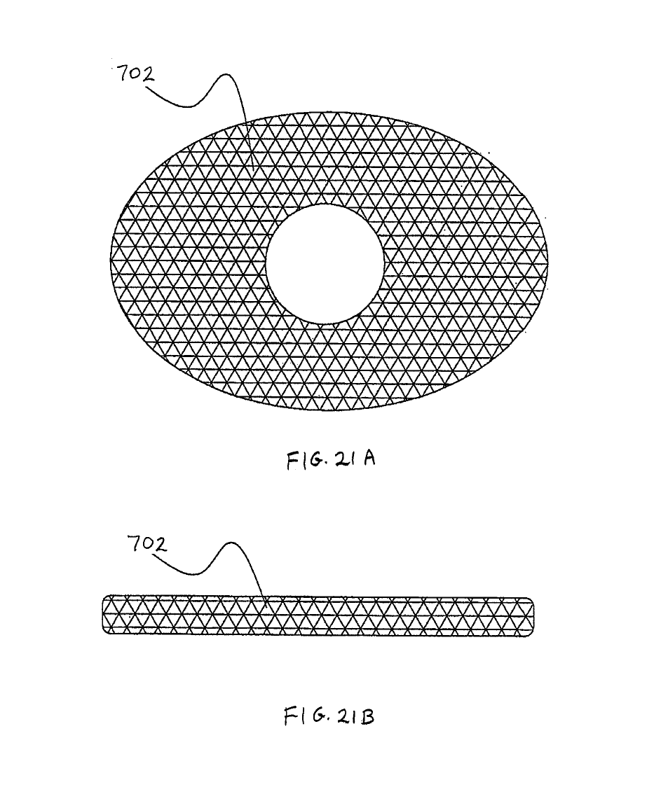

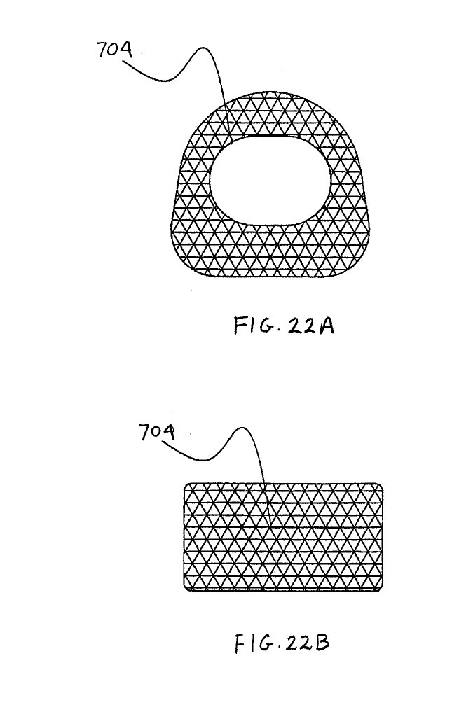

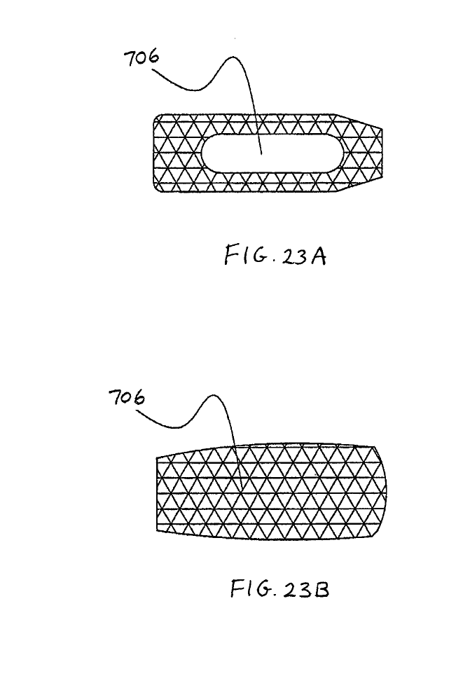

FIGS. 20A-28 show some examples of elastic mesh bodies manufactured according to aspects of the invention. The elastic mesh bodies may be formed in various shapes. FIGS. 20A and 20B show a round elastic mesh body 226 with a central hole. FIGS. 21A and 21B show an elliptical mesh body 702 with a central hole. These two elastic mesh bodies may be used as a bone screw pad as shown in FIGS. 8 and 14. As illustrated in FIG. 19, the mesh bodies can match the complex anatomical surfaces of the spine facets to obtain stable fixation of the facet joints. FIGS. 22A and 22B, and FIGS. 23A and 23B show two specially-shaped elastic mesh bodies with holes, 704 and 706 respectively, that may be used as vertebral interbody fusion cages or interbody filling blocks. For the elastic mesh bodies with holes such as these, in the manufacturing process, the mesh may first be wound on a mandrel of a molding machine and then molded.

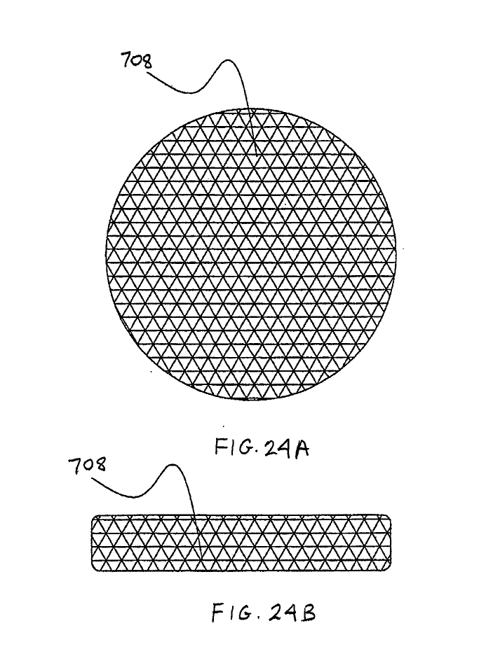

FIGS. 24A-26B show some examples of elastic mesh bodies without holes in them. As shown, the mesh bodies may be round 708, elliptical 710, or square 712. Of course, it will be understood by those skilled in this art that these are only some examples and the shapes can vary based on needs in practical use. For mesh bodies without a hole, in the manufacturing process, the mesh may be rolled up and put directly into a mold.

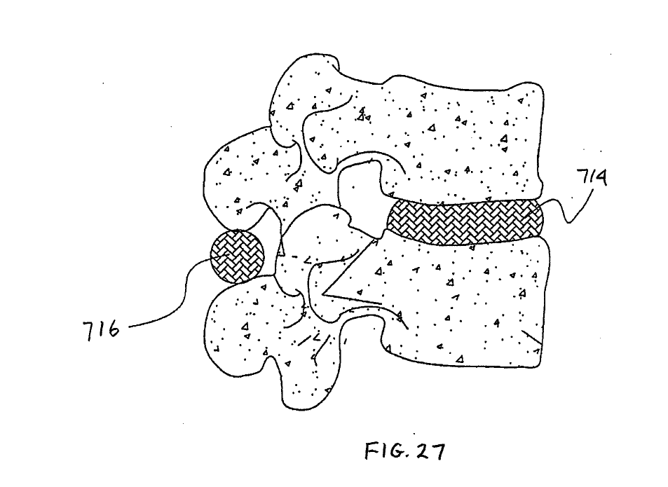

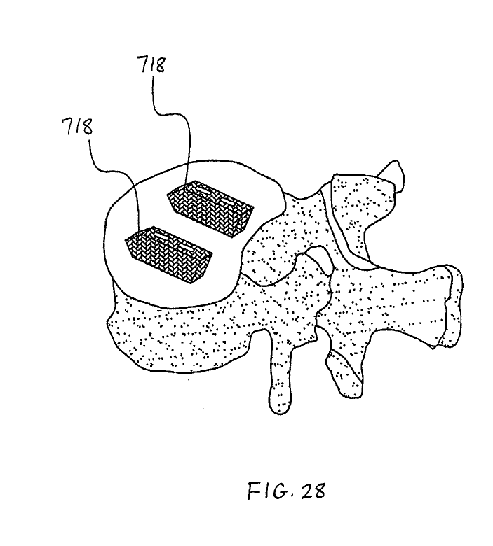

FIGS. 27 and 28 show additional examples of elastic mesh bodies constructed according to aspects of the invention. FIG. 27 shows elastic mesh bodies being used as a synthetic disc 714 and an expander 716 between adjacent spinous processes. FIG. 28 shows a pair of elastic mesh bodies 718, 718 being used as interbody fusion cages or interbody filling blocks. Of course, it will be understood by those skilled in this art that these are only some examples and the elastic mesh bodies may also be used in other suitable applications as well.

While exemplary embodiments of the present invention have been shown and described, modifications may be made, and it is therefore intended in the appended claims to cover all such changes and modifications which fall within the true spirit and scope of the invention.

What is claimed is:

1. An implantable elastic material configured for use with bone implants, the material comprising:
a wire wound in an axially expanded coil form, wherein the expanded coil has been formed into a tight mesh.

2. The material of claim 1, wherein the wire comprises a titanium alloy.

3. The material of claim 1, wherein at least a portion of the wire has a coating selected from the group consisting of an osteogenic inducer, an osteogenic inhibiter, a medicine, or a combination thereof.

4. The material of claim 1, wherein microparticles of a slow release composition are implanted in pores of the material.

5. The material of claim 1, wherein the wire has a diameter of between about 0.1 mm and about 0.5 mm.

6. The material of claim 1, wherein the axially expanded coil has a pitch that is about three times its nominal diameter.

7. A bone screw pad comprising the material of claim 1.

8. A spinous process expander comprising the material of claim 1.

9. A vertebral interbody fusion cage comprising the material of claim 1.

10. A synthetic nucleus pulposus comprising the material of claim 1.

11. A bone filling block used in osteosynthesis comprising the material of claim 1.

12. A manufacturing process comprising the steps of;
winding a wire into a coil;
winding the coil around a work piece;
removing the coil from the work piece; and
compressing the coil into an implantable elastic mesh.

13. The manufacturing process of claim 12, further comprising the step of expanding the coil to a predetermined pitch after it is formed from the wire and before the coil is wound around the work piece.

14. The manufacturing process of claim 13, wherein the predetermined pitch that is about three times the nominal diameter of the coil.

15. The manufacturing process of claim 12, wherein the coil is wound around a plate-shaped work piece.

16. The manufacturing process of claim 12, wherein the coil is first wound in one lateral direction along the work piece, then in the opposite lateral direction, and then these steps are repeated until a mesh of required density is achieved.

17. The manufacturing process of claim 12, wherein the coil is first wound in one lateral direction along the work piece with a first pitch, then in the opposite lateral direction with a second pitch that is about half of the first pitch.

18. The manufacturing process of claim 12, wherein the compressing step comprises winding the coil removed from the work piece around a mandrel.

19. The manufacturing process of claim 12, further comprising the step of coating at least a portion of the wire with a coating selected from the group consisting of an osteogenic inducer, an osteogenic inhibiter, a medicine, or a combination thereof.

20. The manufacturing process of claim 19, wherein the coating step occurs before the wire is wound into a coil.

21. The manufacturing process of claim 19, wherein the coating step occurs after the wire is wound into a coil.

22. The manufacturing process of claim 12, further comprising the step of implanting microparticles of a slow release composition into pores of the implantable elastic mesh.

23. The manufacturing process of claim 12, further comprising the step of forming a bone screw pad with the implantable elastic mesh.

24. The manufacturing process of claim 12, further comprising the step of forming a spinous process expander with the implantable elastic mesh.

25. The manufacturing process of claim 12, further comprising the step of forming a vertebral interbody fusion cage with the implantable elastic mesh.

26. The manufacturing process of claim 12, further comprising the step of forming a synthetic nucleus pulposus with the implantable elastic mesh.

27. The manufacturing process of claim 12, further comprising the step of forming a bone filling block used in osteosynthesis with the implantable elastic mesh.
